(19) United States
(12) Patent Application Publication
(10) Pub. No.: US 2009/0047336 A1
(43) Pub. Date: Feb. 19, 2009

(54) NOVEL FORMULATION OF DEHYDRATED LIPID VESICLES FOR CONTROLLED RELEASE OF ACTIVE PHARMACEUTICAL INGREDIENT VIA INHALATION

(75) Inventors: Zhijun Yang, Kowloon (CN); Wenhua Huang, Kowloon (CN); Chi Sun Wong, Hong Kong (CN); Zhongzhen Zhao, Kowloon (CN)

Correspondence Address: LEYDIG VOIT & MAYER, LTD, 700 THIRTEENTH ST. NW, SUITE 300, WASHINGTON, DC, 20005-3960, US

(73) Assignee: HONG KONG BAPTIST UNIVERSITY, Kowloon (CN)

(21) Appl. No.: 11/840,537

(22) Filed: Aug. 17, 2007

(51) Int. Cl. A61K 31/137; A61K 47/06; A61K 9/127; A61K 9/14; A61P 11/00; A61P 11/06

(52) U.S. Cl. 424/450; 424/489; 514/653; 514/772

(57) ABSTRACT

A new formulation of dehydrated lipid vesicles employs a vesicle preserver and permits the control of release and delivery of active pharmaceutical ingredients into the respiratory system for treatment in particular of asthma. The typical formulation provides controlled release of the active pharmaceutical ingredient from 0% to 100% from 0 to 72 hours after inhalation, changes the systemic administration to topical administration, allows prolonged therapeutic period for one administration, increased stability, with reduced dose, reduced systemic side effects, reduced toxicity.

### FIELD OF THE INVENTION

The present invention relates to lipid vesicles, and in particular to the treatment of asthma and other conditions.

### BACKGROUND OF THE INVENTION

Asthma is a chronic disease of the respiratory system in which the airway discontinuously constricts, with associated inflammation. This causes symptoms such as coughing, wheezing, and shortness of breath with chest tightness. The symptoms of asthma, which range from mild to life threatening, respond to bronchodilators and can usually be controlled with a combination of medication. In the developed countries, asthma has been focused upon because of its rapidly increasing prevalence, affecting up to one in every four urban children, see Lilly CM. Diversity of asthma: Evolving concepts of pathophysiology and lessons from genetics. J Allergy Clin Immunol. 2005; 115 (4 Suppl):S526-31.

Animal models have confirmed a role for A2B antagonists in respiratory inflammation, fibrosis and airway remodelling, as in D. Zeng & R. Polosa (2006) European Respiratory Disease. 2006; 26-27, and the causes of asthma lies in a special type of natural killer cell. That means that asthma is often not treated with the right kind of active pharmaceutical ingredient. For example, natural killer T cells seem to be resistant to the corticosteroids in widely used inhalers, see Cromie, William J. Harvard University Gazette, Harvard News Office, Mar. 16, 2006, Retrieved on Sep. 23, 2006.

Glucocorticoids are the most widely used of the preventive active pharmaceutical ingredients, such as ciclesonide, beclomethasone, budesonide, flunisolide, fluticasone, mometasone, triamcinolone, etc. Using corticosteroids long-term has many side effects, in particular high doses of steroids may cause osteoporosis. Currently long acting β-adrenoceptor agonists, including sustained-release oral albuterol, sameterol, formoterol, and, bambuterol are available. However, the US Food and Drug Administration (FDA) released a health advisory in November 2005; alerting that the use of long-acting β-2 agonists could lead to a worsening of symptoms, indeed to death in some cases. A study says that three common asthma inhalers containing the active pharmaceutical ingredient salmeterol or formoterol may be causing four out of five US asthma-related deaths per year and should be taken off the market, Ramanujan, Krishna (Jun. 9, 2006). Cornell Chronicle Online. Cornell News Service. Retrieved on Sep. 23, 2006.

Bronchodilators are recommended for short-term relief in all patients with asthma. A higher dose of glucocorticoid may be prescribed with a long-acting β-2 agonist, theophylline, leukotriene modifier, or mast-cell stabilizer, for persistent disease. Symptomatic control of wheezing and shortness of breath is generally achieved with a fast-acting bronchodilator. The active pharmaceutical ingredients include selective β-2 adrenergic receptor agonists, such as salbutamol (albuterol), terbutaline, levalbuterol, and bitolterol. There may also be cardiac side effects at higher doses due to β-1 agonist activity, such as elevated heart rate or blood pressure. With the advent of selective agents, these side effects have become less common. Patients must be cautioned against using these medicines too frequently, as with such use their efficacy may decline, producing desensitization resulting in an exacerbation of symptoms which may lead to refractory asthma and death. Older, less selective adrenergic receptor agonist, such as inhaled ephedrine and epinephrine tablets, have also been used. Cardiac side effects occur with these agents at similar rate to albuterol, Hendeles L, Marshik PL, et al. et al. Response to nonprescription epinephrine inhaler during nocturnal asthma. Ann Allergy Asthma Immunol. December 2005;95(6):530-4, and Rodrigo G J, Nannini L J. Comparison between nebulized adrenaline and β-2 agonists for the treatment of acute asthma. A meta-analysis of randomized trials. Am J Emerg Med. March 2006; 24(2):217-22. Their use via injection has declined due to related adverse effects. These are typically provided in pocket-sized, metered-dose inhaler or asthma spacer or nebulizer.

Attempts to formulate active pharmaceutical ingredient in appropriate vehicles for targeted use have often been unsuccessful. Active pharmaceutical ingredient formulated for inhalation seems to be rapidly absorbed, necessitating frequent dosing, which heightens systemic side effects. It may also lead to the mucosal of respiratory tissue damage caused by a repeated use of fluorocarbon propellants, solvents, or other additives necessary for nasal or oral inhalation administration. The aerosol droplets carrying the active pharmaceutical ingredient should avoid multiple dosing while providing a maximum therapeutic benefit. It should also provide a controlled-release of the active pharmaceutical ingredient in the respiratory system, while the active pharmaceutical ingredient should be released continually over an extended period, providing an effective dose on β-2 agonist in the smooth muscle with the minimum amount of active pharmaceutical ingredient. By developing an appropriate formulation vehicle for such therapy, the undesirable side effects accompanying active pharmaceutical ingredient therapy of asthma would be diminished.

Bystrom, K., Nilsson, P. in U.S. RE38407 (2001) describe pain management with liposome-encapsulated analgesic drugs delivered by the pulmonary route to provide local or systemic analgesia. Waldrep, J. C., Knight, V., Black, M. B. in U.S. Pat. No. 5,958,378 (1999) employ about 130˜375 mg/ml of phospholipids for the liposomal aerosol delivery of about 12˜30 mg/ml of a active pharmaceutical ingredient via respiratory system for treatment of diseases. Examples of further liposomal treatments include those for respiratory influenza {Edwards, D. A., Stone, H. A.: US 20050220720A1 (2005)}, tumor {Jin, T., Zarif, L., Mannino, R.: US 2006153217 (2000)}, local respiratory infections and cystic fibrosis {Hersch, E. M., Petersen, E. A., Proffitt, R. T., Bracken, K. R., Chiang, S-M.: U.S. Pat. No. 5,958,449 (1999)}. Liposome-containing two-phase polymer solution, treated with Ca²⁺ or Zn²⁺ etc. cochleate is described in Parmar, M.: US2006 0051406A1 (2006) to achieved efficient delivery of pharmaceutical agents. However, the lipids vesicles are not very stable for storage and producing in a great amount because most of the lipids are easy to disassemble, and the lipids vesicles are in the hundreds nanometer size, which when dispersed in buffer solution must experience Brownian motion, that may lead to the congregation of the liposome vesicle and leakage of the active pharmaceutical ingredient.

Bystrom, K., Nilsson, P., U.S. Pat. No. 6,045,828 (1996) describes a simple mixed powder of lipids and active pharmaceutical ingredient developed for inhalation, which is capable of hydration to form liposomes. In that sense the powder is anhydrous. The manufacturing process of the lipid vesicle powder employs lipids that have a phase transition temperature of below 37° C. Disclosure of phospholipids powders for rapid absorption of the active pharmaceutical ingredient is in Weers, J. G., Tarara, T., Clark, A.: US 20040105820A1 (2004) and in Mezei, M., Hung, O.: U.S. RE38407 (2004].

Radhakrishnan, R.: U.S. Pat. No. 5,049,389 (1991) discloses lipid particle formulations that claim prolonged release of the active pharmaceutical ingredient, improved therapeutic ratio, reduced toxicity, reduced systemic side effects, and stability for several months. The formulation is in particular suitable for treatment of asthma. New steroidal derivatives obtained by modification of corticosteroids with fatty acid esters were incorporated in the lipid portion of liposomes for delivery via inhalation resulting into prolonged steroid retention in the respiratory tract of experimental animals. In the liposomal active pharmaceutical ingredient powder, active pharmaceutical ingredient encapsulated in liposome is homogenized, dispersed into carrier and converted into dry powder by spray drying and/or freeze drying. On inhalation, active pharmaceutical ingredient mixed with lipids get partially rehydrated in the respiratory system and give release active pharmaceutical ingredient. The so-called proliposome is only the mixture of the active pharmaceutical ingredient and lipids, and so it is difficult to maintain the entrapment or encapsulation efficiency because the lipid vesicle may be break or transfigure, and the active pharmaceutical ingredient can leak out from the vesicles during the drying and rehydration processes, no matter what the identity of the active pharmaceutical ingredient is, water soluble or water insoluble. Moreover, the active pharmaceutical ingredient, which once entrapped in the rehydrated liposome vesicle, must wait for the cells to destroy the vesicle to be released.

From above, many problems may be seen remaining unresolved with active pharmaceutical ingredient formulations using the liposomes or proliposomes. These problems relate to the requirement for proper control of release rate.

### OBJECTS OF THE INVENTION

It is the primary object of this invention to provide dehydrated lipid vesicle compositions wherein the active pharmaceutical ingredients can be successfully sequestered within the liposome vesicle without rupture or transfiguring during the drying and rehydration processes, and with controllable particle size, long-term stability, and effective controllable potency of the active pharmaceutical ingredient. A related object of the resulting composition is to allow an administration of low doses of the active pharmaceutical ingredient thus reducing toxicity and systemic side effects and in total providing the desired therapeutic effects.

### SUMMARY OF THE INVENTION

The present invention relates to a novel dehydrated lipids vesicle formulation suitable for the treatment of asthma. In particular, the composition provides efficient control of release of active pharmaceutical ingredient deposited in the respiratory system via small size aerosol particles, and is particularly useful in formulating active pharmaceutical ingredient for inhaled and nebulized inhalation of small aerosol particles.

The first aspect of this invention is to provide the formulation to form the dehydrated lipids vesicles for delivery of various active pharmaceutical ingredient by nebulizer or inhaler into the respiratory system tissue. The dehydrated lipids vesicles formed with uniform and controllable particle size enable the active pharmaceutical ingredient to be entrapped or encapsulated, and are suitable for delivery of active pharmaceutical ingredient to the respiratory system.

The second aspect of this invention is to provide the formulation, the dehydrated lipids vesicles, with high encapsulation efficiencies for encapsulating both water-soluble and water-insoluble active pharmaceutical ingredients suitable for inhalation, with lower toxicity and side effects, allowing the targeting to and release of active pharmaceutical ingredient in a respiratory system tissue, removing need for multiple dosing, and sufficiently stable in dried form for long-term storage.

The third aspect of this invention is to provide control of release in the respiratory system of the active pharmaceutical ingredient from the dehydrated lipid vesicle active pharmaceutical ingredient composition; and provide a process for making the dehydrated lipids vesicles compositions for control of release of active pharmaceutical ingredient delivered by inhalation; and provide a method of treatment of asthma by administering the nebulized or inhaled and inhaled dehydrated liposome vesicle active pharmaceutical ingredient composition.

### BRIEF DESCRIPTION OF THE DRAWINGS

FIG. 1 provides TEM (transmission electron microscope) photographs of dehydrated albuterol lipid vesicles.

FIG. 2 charts the release of albuterol from dehydrated lipid vesicle to buffer solution.

### DETAILED DISCLOSURE OF THE INVENTION

According to the present invention, it has been discovered that albuterol and other active pharmaceutical ingredients may be successfully retained in dehydrated lipid vesicles for control of release in respiratory system tissues when the liposomes are formulated to contain a stabilizer and/or plasticizer such as glycerin. The stabilizers and/or plasticizers act as a vesicle preserver to temporarily maintain the liposome vesicle shapeduring the drying processes of lyophilization or spray drying, and act as a temporary barrier against active pharmaceutical ingredient efflux from the liposomes vesicle. To design the optimal formulation for high active pharmaceutical ingredient load and the control of release of active pharmaceutical ingredient, a number of different formulations were developed, studied, and compared with compositions comprising components of the invention in various amounts and ratios as well as conventional liposomes derived from all kinds of the lipids or their mixtures such as egg, soybean, and synthetic phospholipids.

In one preferred form, the present invention provides a pharmaceutical lipid composition for treatment of asthma by inhalation into the respiratory system, the composition comprisingdehydrated lipid vesicles of pharmaceutically acceptable vesicle preserver, pharmaceutically acceptable lipid component and active pharmaceutical ingredient.

The vesicle preserver is chosen from stabilizers and plasticizers. Plasticizer is a term for describing the function of the pharmaceutical excipients, but there is no common physical chemistry characteristic and chemical structure. As it is common in chemical and pharmaceutical industry, plasticizer is a chemical added to impart flexibility, workability, or stretchability. The most frequently used is glycerol; sorbitol, propylene glycol, sucrose and acacia have been used also. We incorporate by reference the relevant disclosure in Michael E Aulton, (1988), Pharmaceutics: The science of Dosage Dorm Design; international student edition (1996), pp 324, Medical Division of Pearson Professional Ltd, Churchill Livingstone, N.Y.]. Lactose, acacia, etc are used as the stabilizer or stabilizing agents as in pharmaceutical science. For other plasticizers and stabilizers we incorporate by reference the relevant disclosure in Raymond C Rowe, Paul J Sheskey, and Sian C Owen, (2006), Handbook of pharmaceutical Excipients, 5^{th} edition, Pharmaceutical Press, Publications division of the Royal Pharmaceutical Society of Great Britain; and the American Pharmacists Association, USA., and Japan Pharmaceutical Excipients Council (2005) Japanese Pharmaceutical Excipients Directory, YAKUJI NIPPO LIMITED, Tokyo, Japan.

The stabilizers and/or plasticizers can be added before and/or after producing the vesicles.

Suitable candidate vesicle preservers are adipic acid and its derivatives or salts, ascorbic acid and its derivatives or salts, aspartic acid and its derivatives or salts, acetyltryptophan and its derivatives or salts, acetanilide and its derivatives or salts, aminoethy sulfonic acid and its derivatives or salts, alanine and its derivatives or salts, acacia, sodium bisulfite, sodium sulfite, arginine and its derivatives or salts, alginic acid and its derivatives or salts, benzoic acid and its derivatives or salts, isostearic acid and its derivatives or salts, inositol and its derivatives or salts, ethylenediamine and its derivatives or salts, erythorbic acid and its derivatives or salts, lysine and its derivatives or salts, cacao butter, castor wax, xathan gum, xylitol, citric acid and its derivatives or salts, glycine and its derivatives or salts, glycerin and its derivatives, gluconic acid and its derivatives or salts, glutamic acid and its derivatives or salts, creatinine, diisopropanolamine and its derivatives, diethanolamine and its derivatives, cyclodextrin, cystine, cysteine, dibutylhydroxytoluene, tartaric acid and its derivatives or salts, sucrose esters of fatty acids, stearic acid and its derivatives or salts, gelatin, lanolin, cetanol, gelatin, hydrolyzed gelatin, shellac, D-sorbitol, sorbitan esters of fatty acid, sorbica acid and its derivatives or salts, thioglycolic acid and its derivatives or salts, potassium thiocyanate, sodium thiomalate, thymol, medium chain fatty acid triglyceride, dextran, dextrin, vitamin E, calcium D-saccharate, tocopherol and its isomer, trometamol, nicotinamide, lactic acid and its derivatives or salts, lactose, carbamide, white soft sugar, histidine and its derivatives or salts, hydroxypropylcellulose, hyroquinone, phenylalanine, phenacetin, glucose, fumaric acid and its derivatives or salts, propylene glycol, heparin sodium, povidone, maleic acid and its derivatives or salts, malonic acid and its derivatives or salts, mannitol, methionine, sodium lauryl sulfate, malic acid and its derivatives or salts, hydrogenated oil, sesame oil, karion 83, diethylenetriaminepentaacetic acid and its derivatives or salts, dioctyl sodium sulfosuccinate, polydimethylsiloxane-silicone dioxide mixture, sorbitan esters of fatty acid, triacetin, castor oil, diethyl/dibutyl phthate, butylphtalylbutylglycolate, propylene glycol (1,2-propane diol), propylene glycol esters of fatty acids, polysorbate, polyoxyethylene polyoxypropylen glycol, macrogol, isopropyl myristate, contton seed oil-soybean oil mixture, glyceryl monostearate, isopropyl linoleate, petrolatum, etc. These stabilizers and/or plasticizers can be used as mixtures.

Preferred candidates similar to glycerin are selected from the following list:
2-pyrrolidone,
acetytributyl citrate,
acetytriethyl citrate,
benzyl benzoate,
butylphthalylbutylglycolate
cellulose acetate phthalate compatible,
chlorbutanol,
cotton seed oil-soybean oil mixture,
dextrin,
dibutyl phthalate,
dibutyl sebacate,
diethyl phthalate,
dimethyl phthalate,
dioctyl adipate,
dioctyl phthalate,
D-sorbitol,
gelatin,
glycerin,
derivatives of glycerin,
glyceryl monostearate,
hypromellose phthalate compatible
isopropyl linoleate,
isopropyl myristate,
karion 83,
macrogol,
mannitol,
mineral oil and lanolin alcohols,
palmitica acid
phytosterol
polyethylene glycol,
polymethacrylate compatible,
polyoxyethylene polyoxypropylen glycol,
polysorbate,
polyvinyl acetate phthalate
propylene glycol,
sesame oil,
sobitol
stearic acid and its derivatives or salts,
triacetin,
tributyl citrate
triethanolamine
triethyl ctrate
and mixtures thereof.

Preferred candidates similar to lactose are selected from the following list:
acacia,
acetanilide and its derivatives or salts,
acetyltryptophan and its derivatives or salts,
adipic acid
agar
alanine and its derivatives or salts,
albumin
alginic acid and its derivatives or salts,
alginine hydrochloride
aluminum hydroxide gel
aluminum stearate
aminoethyl sulfonic acid and its derivatives or salts,
arginine and its derivatives or salts,
ascorbic acid and its derivatives or salts,
ascorbyl palmitate
aspartic acid and its derivatives or salts,
bentonite
benzalkonium chloride
benzethonium chloride
benzoic acid and its derivatives or salts,
butylated hydroxytoluene
cacao butter,
calcium D-saccharate,
carbamide,
carbazochrome sodium sulfonate
carboxymethylcellulose and it salts
carboxyvinyl polymer
carmelose calcium
carmelose sodium
carrageenan
casein peptone
castor oil,
castor wax,
cellulose acetate phthalate compatible,
ceratonia
cetanol,
chlorbutanol,
citric acid and its derivatives or salts,
colloidal silicon dioxide
cotton seed oil-soybean oil mixture,
creatinine,
cyclodextrin,
cysteineand and its derivatives or salts,
cystine,
dextran, dextrin,
dextrin,
dibutyl phthalate,
dibutyl sebacate,
dibutylhydroxytoluene,
diethanolamine and its derivatives,
diethyl phthalate,
diethyl/dibutyl phthate, butylphtalylbutylglycolate,
diethylenetriaminepentaacetic acid and its derivatives or salts,
diisopropanolamine and its derivatives,
dimethyl phthalate,
Dioctyl adipate,
dioctyl sodium sulfosuccinate,
disodium glycyrrhizinate
D-sorbitol,
edetates
erythorbic acid and its derivatives or salts,
ethylcellulose
ethylene glycol palmitostearate
ethylenediamine and its derivatives or salts,
fumaric acid and its derivatives or salts,
fructose
gelatin,
derivatives or salts of gluconic acid,
glucose,
derivatives or salts of glutamic acid,
glycerin and its derivatives such as glycerin monostearate,
glyceryl monostearate,
glycine and its derivatives or salts,
guar gum
heparin sodium,
histidine and its derivatives or salts,
hydrated silicon dioxide
hydrogenated oil,
hydrolyzed gelatin,
hydroxypropyl cellulose,
hypromellose
hyroquinone,
inositol and its derivatives or salts,
invert sugar
isopropyl linoleate,
isopropyl myristate,
isostearic acid and its derivatives or salts,
karion 83,
lactic acid and its derivatives or salts,
lactose,
lanolin,
lecithin
lysine and its derivatives or salts,
macrogol,
magnesium aluminum silicate
maleic acid and its derivatives or salts,
malic acid and its derivatives or salts,
malonic acid and its derivatives or salts,
mannitol,
medium chain fatty acid triglyceride,
methionine,
microcrystalline cellulose and its derivatives
mineral oil and lanolin alcohols
monoethanolamine
nicotinamide,
pectin
petrolatum,
phenacetin,
phenylalanine,
polacrilin potassium
polydimethylsiloxane-silicone dioxide mixture,
polyoxyethylene polyoxypropylen glycol,
polysorbate,
polyvinyl alcohol
potassium chloride
potassium thiocyanate,
povidone,
propylene gallate
propylene glycol (1,2-propane diol),
propylene glycol alginate,
propylene glycol esters of fatty acids,
propylene glycol,
raffinose
sesame oil,
schizonepeta spike
shellac,
silicic acid
sodium acetate,
sodium alginate,
sodium bisulfite,
sodium borate,
sodium carbonate
sodium caprylate
sodium chloride,
sodium lauryl sulfate,
sodium stearyl fumarate,
sodium sulfite,
sodium thiomalate,
sorbica acid and its derivatives or salts,
sorbitan esters of fatty acid,
sorbitan esters of fatty acid,
sorbitol
stearic acid and its derivatives or salts, gelatin,
stearyl alcohol
sucrose esters of fatty acids,
sulfobutylether β-cyclodextrin
sulfur
tartaric acid and its derivatives or salts,
thioglycolic acid and its derivatives or salts,
thymol,
tocopherol and its isomer,
trehalose,
triacetin,
trometamol,
vitamin E,
white soft sugar,
white wax,
xanthan gum,
xylitol,
zinc acetate
zinc chloride
and mixtures thereof.

The ratio of the stabilizers and/or plasticizers to the lipids is suitably from 0.1 to 40 mole % of the stabilizers and/or plasticizers; from 99.9 to 60 mole % of the lipids. Preferably the ratio of plasticizer to the lipid is 1:5 to 1:50, such as about 1:10.

The active pharmaceutical ingredient is usually selected from the group consisting of ephedrine, ephedrine hydrochloride, albuterol, theophyline, salbutamol sulfate, salmefamol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rimiterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredient, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropium bromide, cromolyn sodium, nedocromil or their respective pharmaceutically acceptable salts or esters, alone or in combination.

The active pharmaceutical ingredients that are the medicines which are used for treatment of disorders in the respiratory system, for example, the albuterol, terbutalin etc are the medicines used in asthma. We incorporate by reference the relevant disclosure in Bertram G. Katzung, (2001), Basic & Clinical Pharmacology, 8^{th} edition, Medical Publishing Division, McGraw-Hill Companies, Inc. USA.

The mole ratio of active pharmaceutical ingredient to the lipid component is usually from 0.1% to 200%.

A preferred comopsition employs albuterol present in amount between 0.1 to 300 mg/ml of dehydrated lipid vesicles composition.

The composition is preferably one which can be aerosolized into particles predominantly smaller than mass median aerodynamic diameter 10 μm.

In a related aspect, the invention provides a method of treating asthma by inhalation route of administration to a person in need of such treatment a therapeutically effective amount of plasticized lipid composition consisting essentially of an active pharmaceutical ingredient, a vesivle preserver selected from a plasticizer, a stabilizer and mixtures thereof, and lipid component aerosolized into aerosol particles having a mass median aerodynamic diameter smaller than 10 μm and providing a slow or controlled release of the active pharmaceutical ingredient in the respiratory system.

In the method the pharmaceutical stabilizers and/or plasticizers lipid composition form dehydrated lipid vesicles preferably comprising 0.1 to 40 mole % of the stabilizers and/or plasticizer, 99.9 to 60 mole % of lipids, and the active pharmaceutical ingredient is from 0.01 to 200 mole % to the lipids.

For the method the active pharmaceutical ingredient can be selected from the group consisting of ephedrine, ephedrine hydrochloride, albuterol, theophyline, salbutamol sulfate, salmefamol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rimiterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredientne, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropium bromide, cromolyn sodium, nedocromil or their respective pharmaceutically acceptable salts or esters, alone or in combination.

When albuterol is present, the amount is preferably from 0.1 to 300 mg/ml of dehydrated lipid vesicle composition.

In a further aspect, the invention provides an inhalation method for treatment of respiratory system diseases by treating a person in need of such treatment with a therapeutically effective amount of aerosolized dehydrated lipid vesicle composition consisting essentially of a active pharmaceutical ingredient and lipid components aerosolized into particles predominantly smaller than 10 μm mass median aerodynamic diameter by the inhalation route of administration.

The active pharmaceutical ingredient is suitably selected from the group consisting of ephedrine, ephedrine hydrochloride, albuterol, theophyline, salbutamol sulfate, salmefamol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rimiterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredientne, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropium bromide, cromolyn sodium, nedocromil or their respective pharmaceutically acceptable salts or esters, alone or in combination.

Where albuterol is present the amount is usually from 0.1-300 mg/ml.

In a yet further aspect, the invention provides a process of preparing a suspension of inhaleable or nebulizeable aerosol particles of sizes predominantly smaller than 10 μm being particles of dehydrated liposome vesicles, the process comprising providing dehydrated liposome vesicles having sizes less than 10 μm in an aqueous suspension; and inhaling or nebulizing the suspension under conditions which produce aerosol particles of mass median aerodynamic diameter predominantly smaller than 10 μm.

The lipid particle comprises dehydrated lipids vesicles and/or micelle not larger than 1.0 μm, which composition for treatment of asthma consisting essentially of lipid components and an active pharmaceutical ingredient or its salt or ester, suitable for delivery by inhalation into the respiratory system.

Methods of Dehydrated Lipid Vesicles Formation

The dehydrated lipids vesicles of the invention can be prepared by any of the standard methods for preparing and sizing liposomes, but formulating with lipids, stabilizers and/or plasticizers and active pharmaceutical ingredients in the beginning. But that is only the liposome vesicles. Before the liposome vesicles are formed, the stabilizers and or plasticizers may be added in/with the lipid solutions to form the lipid mixed liposome vesicles. The methods for preparing the liposomes include hydration of lipid films, solvents injection, reverse-phase evaporation and vesicular phospholipids gels methods, see Brandl, M., Bachmann, D., Reszka, R., and Drechsler, M. Liposomale Zubereitung, ihre Herstellung und ihre Verwendung. DE 44 30 592.3 (filed Aug. 18, 1995). PCTWO 96/05808 and see Brandl, M., Tard, C., Drechsler, M., Bachmann, D., Reszka, R., Bauer, K. H., et al. (1997). Adv. Drug Deliv. Rev., 24, 161, which are incoproated herein by specific reference. Also incoporated by reference is the detailed information in Ann. Rev. Biophys. Bioeng. 9:467 (1980). Reverse-phase evaporation vesicles (REVs) prepared by the reverse-evaporation phase method is described in U.S. Pat. No. 4,235,871, incorporated hereby by reference. The preparation of multilamellar vesicles (MLVs) by thin-film processing of a lipid film or by injection technique is described in U.S. Pat. No. 4,737,923, incorporated by reference. In the two later procedures, which are generally preferred, a mixture of liposome-forming lipids dissolved in a suitable solvent is evaporated in a vessel to form a thin film, which is covered by an aqueous buffer solution. The lipid film hydrates to form MLVs, typically with sizes between about 0.1 to 10 μm. The REVs or MLVs are further treated to produce a suspension of smaller, substantially homogeneous liposomes, in the 0.02-2.0 μm size range, preferably in 0.2-0.4 μm range. One effective sizing method involves extruding an aqueous suspension of the liposomes through a polycarbonate membrane or asymmetric ceramic filter having a selected uniform pore size, see Ann. Rev. Biophys. Bioeng, 9:467 (1980), and U.S. Pat. No. 4,737,323, incorporated by reference. The pore size of the polycarbonate membrane is near to the size of the vesicles. Thus, the size of the vesicles was typically controlled to be from 20 to 5000 nm. Alternatively, the REVs or MLVs can be treated by sonication or extrusion to produce small unilamellar vesicles (SUVs) which are characterized by sizes 0.02-0.07 μm. Another preferred method for producing SUVs is by homogenizing MLVs, using a conventional high pressure homogenizer of the type used commercially for milk homogenization. Here the MLVs are cycled through the homogenizer, with periodic sampling of particle sizes to determine when the MLVs have been substantially converted to SUVs. The active pharmaceutical ingredient is encapsulated in the liposomes by using for example the procedure described in U.S. Pat. No. 4,752,425, incorporated by reference. After the liposome vesicles are formed, some pharmaceutical excipients which may act as aerosol carriers, more so the stabilizers and or plasticizers, can be added in the liposome solution, and by immediately to cooling to −50° C. the shape and size of liposome vesicles is fixed, then lyophilizing the solid dispersing cake.

After the moisture in the cake has been dried, the dehydrated lipids vesicles are formed with the active pharmaceutical ingredient entrapped in the dehydrated lipid vesicles composed of the stabilizers and/or plasticizers but without an inner water phase in the lipid vesicles.

Conventional and Dehydrated Lipids Vesicle

Conventional liposomes are liposomes which contain pure lipids, while the dehydrated lipids vesicles of the present invention are liposomes which do not contain inner water but are formed by lipids and stabilizers and/or plasticizers and active pharmaceutical ingredient or, alternatively by amphipathic lipid components. The stabilizers and/or plasticizers act as vesicle preservers, and serve to stabilize the lipid vesicles and maintain the shape of the lipid vesicles without water during lyophilization or spray drying.

Both conventional and dehydrated lipids vesicles can be formed by a variety of standard methods from a variety of vesicle-forming lipids. For the conventional liposomes these lipids include dialiphatic chain lipids, such as phospholipids, diglycerides, dialiphatic glycolipids; the stabilizers and/or plasticizers are not specialized to keep the shape of the liposome vesicles thereof. The various lipid components are present in an amount between about 99.9-60 mole % of the total non-aqueous lipid components in the liposomes; stabilizers and/or plasticizers are present in amounts between 0.1-40 mole %. The active pharmaceutical ingredient encapsulated in both kinds of liposomes is in amounts of 0.01-200 mole % to the lipids. The dehydrated lipids vesicles are the products which are prepared by lyophilization or spray dry the conventional liposome which are formed with lipids and stabilizers and/or plasticizers acting to preserve lipid vesicles.

Lipids for the present invention include but are not limited to trimethylammonium-propane (TAP), phosphatidylcholine (PC) including their mixture such as egg phosphatidylcholine (EPC) and lysophosphatidylcholine (LPC), phosphatidylethanolamine (PE), phosphatidylinositol (PI), phosphatidylserine (PS), phosphatidic acid (PA) and phosphatidyl glycerol (PG), and their derivatives or mixtures. These lipids may be fully saturated or partially saturated. They may be naturally occurring or synthetic.

DOPC and DOTAP are preferred lipids. Lipids are a major component of biological membranes, along with phospholipids, glycolipids, cholesterol and proteins. Lipids may be classified in many different ways, and can be subdivided into fatty acids and their derivatives (e.g. DOPC and DOTAP), triacylglycerols, wax esters phospholipids (phosphoglycerides e.g. DOPC and sphingomyelin), sphingolipids (molecules other than sphingomyelin that contain the amino alcohol sphingosine), isoprenoids (molecules made up of repeating isoprene units, a branched five carbon hydrocarbon). Refer to Trudy McKee and James RMcKee: (2003), Biochemistry: the Molecular Bassis of Life, third edition; international edition, pp 332, McGraw-Hill Companies, Inc., (2003)

Following, there is a list of lipids which are similar to DOPC
Phosphatidylcholine (PC)
1,2-Diacyl-sn-Glycero-3-Ethylphosphocholine (EPC)-(Phospholipid)
Phosphatidylethanolamine (PE)
Phosphatidylserine (PS)
Phosphatidylinositol (PI & PIP's)
bis(Monoacylglycero) Phosphate
Phosphatidic Acid (PA)
Phosphatidylglycerol (PG)
Cardiolipin (CA)
Diacylglycerides (DG)
Cholesterol (Plant-Derived)
   and similar to DOTAP:
1,2-Diacyl-3-Trimethylammonium-Propane (TAP)-(Diol)
1,2-Diacyl-3-Dimethylammonium-Propane (DAP)-(Diol)
DC-Cholesterol (DC-Chol)-(Sterol)
Dimethyldioctadecylammonium Bromide (DDAB)-(Alkyl Amine)

The dehydrated lipids vesicles composition may be formulated to include minor amounts of fatty alcohols, fatty acids, and/or stabilizers and/or plasticizers with the proviso that these minor lipid components do not significantly reduce the binding affinity of the liposomes for mucosal or respiratory system tissue, are substantially unsaturated, are not toxic or irritating and co-controlling or adjusting the release properties of the active pharmaceutical ingredient from the dehydrated lipids vesicles when it rehydrates or contacts with water or any buffer solutions in vitro or/and in situ or/and in vivo.

Preparation of Dehydrated Lipid Vesicle Composition

According to the present invention, it has been discovered that albuterol or other active pharmaceutical ingredient may be successfully maintained in lipid vesicle shape and with retention of the liposome vesicle during and after the processes of lyophilization or spray dray and rehydration, and successfully retained in liposomes vesicles for delayed release, when the liposomes are formulated to contain a high percentage of vesicle preserver stabilizers and/or plasticizers, such as glycerin and sucrose esters of fatty acids, typically from 0.1˜40 mole.

According to one aspect of the invention, it has been discovered that the active pharmaceutical ingredient/lipid/stabilizers and/or pasticizers composition of the invention has much improved properties such as lesser leakage active pharmaceutical ingredient from the liposome vesicles, decreased toxicity and side effects, controllable release, improved solubility, high encapsulation, active pharmaceutical ingredient release at the target organ, absence of need for multiple dosing, extended stability in that it can be stored long-term in dried form without significant leakage of the active pharmaceutical ingredient from the lipids vesicles on rehydration, and may be nebulized or inhaled to provide a homogeneous mixture of aerosol particles having mass median aerodynamic diameter smaller than 10 μm.

To achieve all the above advantages, the current invention combines the lipid components including stabilizers and/or plasticizers, providing the hydrophilic group, and the active pharmaceutical ingredient to be formulated to provide a new, highly efficient liposomal composition for formulation of active pharmaceutical ingredient. The composition is engineered for increased active pharmaceutical ingredient loading and a controllable release rate of the active pharmaceutical ingredient in the respiratory system tissue. It also provides a means to solubilize the active pharmaceutical ingredient and incorporate them in such liposomal composition. Further, the formulation can be easily sterilized thus meeting an important requirement for pharmaceutical preparations, and It is also stable and suitable for long-term storage.

The dehydrated lipids vesicles compositions containing active pharmaceutical ingredient may further contain any suitable pharmaceutically acceptable additive, diluent and/or excipient. Examples of such additives, diluents or excipients, such as sodium or potassium chloride, mono or dibasic sodium phosphates in hydrated or dehydrated form, water, saline, etc, are not intended to limit the scope of this invention and may be used in any amount needed or necessary which is pharmaceutically acceptable for inhalation formulations. Pharmaceutically acceptable stabilizers and/or plasticizers and excipients can be used in the formulation. While stabilizers and/or plasticizers are preferred, the composition is not restricted to the particular glycerin, and any other suitable stabilizers and/or plasticizers can be adopted which are commonly used and pharmaceutically acceptable in pharmaceutical formulations.

Buffer used in the preparation of the dehydrated lipids vesicles may be any buffer chosen from the group of citrate, carbonate, bicarbonate, acetate, Tris, glycinate, cacodylate, maleate, and such other, and preferably phosphate buffered saline of pH 7.4.

Any organic solvent such as lower alcohols, dimethoxyethane, dioxane, tetrahydrofuran, tetrahydropyran, diethylether, acetone, dimethylsulfoxide (DMSO), dimethylformamides (DMF), and halogenated hydrocarbons, such as Freon, acetonitrile, or mixtures thereof, preferably chloroform/methanol can be used in the process of generation of liposomes.

The preferred method of preparation of dehydrated lipids vesicles comprises:
(1) mixing stabilizers and/or plasticizers, and active pharmaceutical ingredient in dry form, in amounts from 0.1-40 mole % of stabilizers and/or plasticizers to the lipids and 0.1-200 mole % of active pharmaceutical ingredient;
(2) dissolving the mixture in a suitable volume of an organic solvent, preferably in ethanol;
(3) injecting the ethanol solution into a suitable volume of the buffer at pH 5. 6-7.6, then go to (5); or, repeatedly drying obtained solution under nitrogen and/or vacuum, and/or, lyophilizing the dry film for suitable minutes, at a suitable temperatures;
(4) resuspending the residue in a suitable volume of buffer at pH 5.6-7.6 preferably in the phosphate-buffered saline, pH 7.4;
(5) forming the liposomes by shaking, stiring or sonication, solvent injection or any other suitable method;
(6) sizing the liposomes by extrusion, or by other methods; and
(7) sterilizing the liposomes using suitable and acceptable methods for sterilization of the liposome vesicles formulations;
(8) lyophilizing or spray-drying the liposome vesicles formulations to form the dehydrated lipids vesicles formulations.

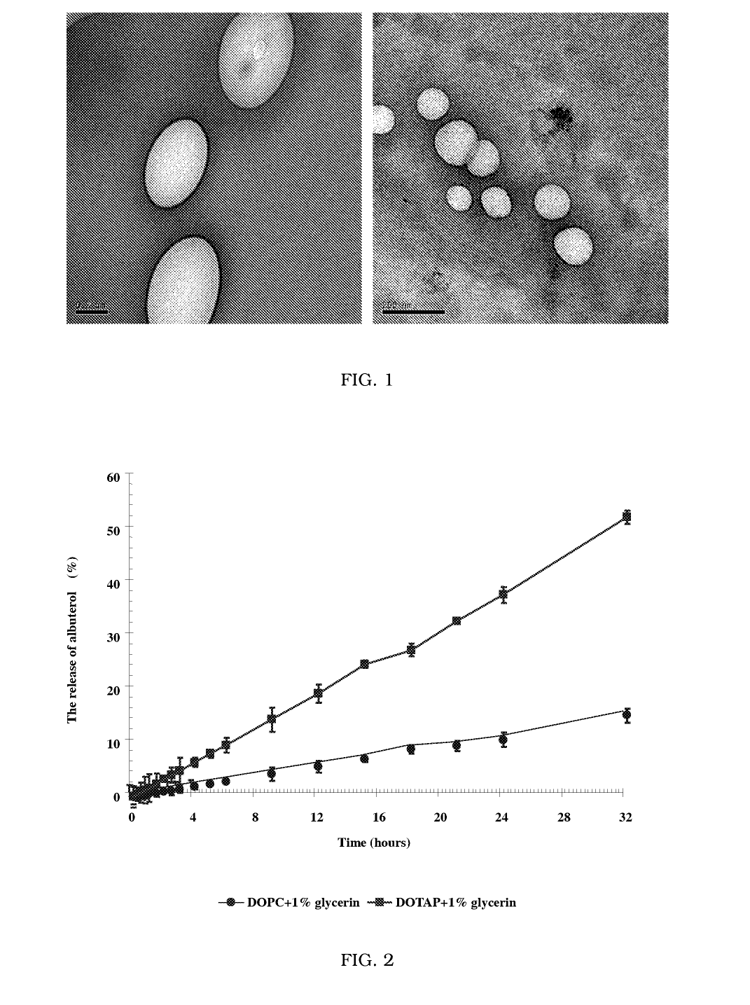

FIG. 1 provides images of TEM pictures of albuterol dehydrated lipid vesicles prepared by such a method.

Methods of preparing the composition of the invention are not limited to those named above, but other methods of dehydrated lipid vesicles preparation such as solvent injection, thin film hydration, dehydration-rehydration, and reverse evaporation are equally suitable.

The size of the preferred dehydrated vesicles is from 20-5000 nm. The moisture content is typically less than 15% by weight, preferably less than 10% by weight, more preferably less than 9% of the powder.

The amounts by weight of the components in the products are normally as follows:

| | range of amounts for | preferred range | most preferred |
|---|---|---|---|
| | commercial product | of amounts | ranges |
| plasticizer | 0.1~99.9% | 1~99% | 10~80% |
| stabilizer | 0.1~99.9% | 1~99% | 10~80% |
| lipid | 0.1~99.9% | 1~99% | 10~80% |
| active | 0.1~99.9% | 1~99% | 10~80% |
| pharmaceutical | | | |
| ingredient | | | |

The powder will be used to prepare the powder or liquid aerosol to treatment the disorder of the respiratory system.

Therapeutic Applications

The control of release is very important for successful active pharmaceutical ingredient delivery to respiratory system. A list of factors which are known to effect a deposition of inhaled particles into respiratory system include characteristics of the aerosol or its environment, characteristics of the respiratory system structure, characteristics of the inhalant, and characteristics of the breathing pattern. FIG. 2 shows release profiles of two kinds of dehydrated lipid vesicles formulations.

The therapeutic applications and advantages of the aerosolizing dehydrated lipids vesicles and micelles in small particles are numerous. Inhaled aerosolized small particles will deposit an active pharmaceutical ingredient encapsulated in dehydrated lipids vesicles in the respiratory tissue in high enough amounts to allow minimal daily dosing with maximal effect extended over a period of time by controlled release. Controlled release of the active pharmaceutical ingredient from the dehydrated lipids vesicles is expected to prolong the therapeutic activity after each administration, reduce the frequency of administration, further improve the ratio of localized-to-systemic effects, and provide increased and extended local therapeutic effect in the respiratory systems.

In one aspect of this invention, the lipid vesicles containing active pharmaceutical ingredient are suspended or/and dissolved or diluted with 0.9% sterile saline and the suspension placed, after mixing, in a nebulizer and the aerosol is breathed until there is no more liquid in the nebulizer or inhaler. The examples providing the data and evaluating the new inhalation composition in this application primarily use the anti inflammatory active pharmaceutical ingredient albuterol, for inhalation of nebulized or inhaled aerosol particles into the deep respiratory system. The scope of the invention is not limited to albuterol as an active pharmaceutical ingredient.

Examples of the classes of compounds to be used in this composition administered through inhalation therapy include, but are not limited to bronchodilators, such as metaproterenol sulfate, aminophylline, terbutaline, albuterol, theophyline, ephedrine, isoproterenol, bitolterol, pirbuterol, adrenaline, norepinephrine, procaterol, and salmeterol; antiinflammatory active pharmaceutical ingredient, such as albuterol, dexamethasone, prednisolone, hydrocortisone, fluoromethasone, medrysone, fluticasone, triamcinolone, and flunisolide.

For inhalation, the delivery is achieved by (a) aerosolization of a dilute aqueous suspension by means of a pneumatic or ultrasonic nebulizer, (b) spraying from a self-contained atomizer using an air; solvent with suspended, dried the dehydrated lipids vesicles in a powder, (c) spraying dried particles into the respiratory systems with a propellant or (d) delivering dried liposomes as a powder aerosol using a suitable device, provided that the aerosol particles generated by any of the above means are in the size range from 0.02-20 μm. The composition of the current invention typically has high encapsulation values, good stability, and extended shelf-life.

The following examples illustrate methods of preparing dehydrated lipids vesicles for formulation of active pharmaceutical ingredient and using these dehydrated lipids vesicles for treatment of interstitial respiratory system disease. These examples are in no way intended to limit the scope of the invention.

### EXAMPLES OF THE INVENTION

Materials

Albuterol [2-tert-butylamino-1-(4-hydroxy-3-hydroxy-methylphenyl) ethanol] was purchased from the Yabang chemical industry Corporation (Changzhou, Jiangsu, China). EPC, SPC, DOTAP (1,2-dioleoyl-3-trimethylammonium propane), DOPC (dioleoyl phosphatidylcholine), DSPC (distearoyl phosphotidylcholine), sphingomyelin, ceramide-1-phosphocholine, and DMPC ((dimyristoyl phosphatidylcholine) were purchased from Avanti Polar Lipids, Inc (USA). Glycerin and other stabilizers or plasticizers and solvents such as chloroform [RDH(Riedel-de Haën)] was purchased from Kou Hing Hong Scientific Supplies Ltd., tris (hydroxymethyl) aminomethane (Tris) (Sigma) was purchase from Tin Hang Technology Ltd., and monobasic potassium phosphate (Amersham Biosciences) was purchased from Amersham Biosciences China Ltd. All of the solvents, solutions and chemicals were of analytical reagent grade.

### Example 1

Preparation of Liposomes By Thin Film Hydration

Aqueous multilamellar vesicles (MLV) were prepared by conventional lipid membrane hydration method and subsequent small unilamellar vesicles (SUV) were produced by extrusion. Lipid and albuterol were dissolved in chloroform and methanol respectively, and then the solutions were mixed with the indicated molar ratios of lipid and albuterol. The mixture was dried into a homogeneous lipid membrane under a stream of nitrogen gas, and then under vacuum overnight to remove any residual organic solvents. This lipid membrane was hydrated in 10 mM Tris-buffered isosmotic saline buffer solution (10 mM Tris, 137 mM sodium chloride, and pH 7.4 at 25° C.). The final concentration of the lipid was controlled at 5˜20 mg/mL. Then the mixture was maintained at 80° C. (over the transition temperatures of all lipids) for 60 minutes to anneal the liposome structure. During annealing, it was stirred 3 times with vortex at the beginning, middle, and ending time points respectively, and each time was stirred continually for 5 minutes. The resulting MLV was then extruded through Whatman 100 nm polycarbonate filters (Nuclepore, Pleasanton, Calif.) with a diameter of 25 mm, using a 10 mL extruder (Lipex Biomembranes Inc., Vancouver, Canada) with several cycles of extrusion till the sizes were within 50˜200 nm.

Determination of Encapsulation Efficiency

150 μL of freshly prepared liposomal sample was centrifuged with an Avanti J-E centrifuge (type JA-20, 17400×g, 6° C., and 20 min) through Microcon Y-10 Centrifugal Filter Devices (Millipore) with a cut-off value of 10,000 Dalton. The concentration of albuterol in the centrifuged solution was determined spectrophotometrically at 276 nm. This concentration represented the concentration of albuterol in the continuous phase of the liposome (non-encapsulated albuterol). The spectrophotometrical method was also used with the determining of the total concentration of albuterol including in the dispersed phase and the continuous phase of liposome. Absolute ethanol was added into the liposome suspension to disrupt the liposome completely and the encapsulated albuterol was completely dissolved in the solution. The absorbencies were measured by a UV-VIS Varian Cary 50 Tablet instrument equipped with a thermostated quartz cell at the wavelengths of 278 nm. The encapsulation efficiency (EE) was calculated with the formula:

EE(%)=(Cₜₒₜₐₗ−C_{free})/Cₜₒₜₐₗ×100

where C_{free} is the concentration of unencapsulated albuterol in the continuous phase of liposome dispersion/solution, and Cₜₒₜₐₗ is the total concentration of albuterol in the liposome dispersion/solution. The encapsulation efficiencies were 20˜30%.

### Example 2

Preparation of Liposomes by Solvent Injection Technique

A mixture of partially DOPC or DOTAP, and active pharmaceutical ingredient (albuterol, 0.04 mmol) in the mole ratio of 1:2 was dissolved in 4 ml of ethanol. Liposomal albuterol dispersion was formed by injecting the lipid/active pharmaceutical ingredient/ethanol solution into 50 ml of the phosphate buffered saline pH 7.4. Liposomes thus formed were extruded through a 0.4 or a 0.2 μm polycarbonate membrane to produce uniform size liposome vesicles distribution.

Determination of Encapsulation Efficiency

150 μL of freshly prepared liposomal sample was centrifuged with an Avanti J-E centrifuge (type JA-20, 17400×g, 6° C., and 20 min) through Microcon Y-10 Centrifugal Filter Devices (Millipore) with a cut-off value of 10,000 Dalton. The concentration of albuterol in the centrifuged solution was determined spectrophotometrically at 276 nm. This concentration represented the concentration of albuterol in the continuous phase of the liposome (non-encapsulated albuterol). The spectrophotometrical method was also used with the determining of the total concentration of albuterol including in the dispersed phase and the continuous phase of liposome. Absolute ethanol was added into the liposome suspension to disrupt the liposome completely and the encapsulated albuterol was completely dissolved in the solution. The absorbencies were measured by a UV-VIS Varian Cary 50 Tablet instrument equipped with a thermostated quartz cell at the wavelengths of 278 nm. The encapsulation efficiency (EE) was calculated with the formula:

EE(%)=(Cₜₒₜₐₗ−C_{free})/Cₜₒₜₐₗ×100

where C_{free} is the concentration of unencapsulated albuterol in the continuous phase of liposome dispersion/solution, and Cₜₒₜₐₗ is the total concentration of albuterol in the liposome dispersion/solution. The encapsulation efficiencies were 20˜30%.

### Example 3

Preparation of Liposomes by Vesicular Phospholipid Gels.

1 ml of albuterol saturated water, 0.1 g of glycerin and 2 g of SPC was mixed by high speed homogenizer under 50° C. till forming gels; and then the gels diluted in 100 ml of the phosphate buffered saline pH 7.4 to form the liposome vesicles.

Determination of Encapsulation Efficiency 150 μL of freshly prepared liposomal sample was centrifuged with an Avanti J-E centrifuge (type JA-20, 17400×g, 6° C., and 20 min) through Microcon Y-10 Centrifugal Filter Devices (Millipore) with a cut-off value of 10,000 Dalton. The concentration of albuterol in the centrifuged solution was determined spectrophotometrically at 276 nm. This concentration represented the concentration of albuterol in the continuous phase of the liposome (non-encapsulated albuterol). The spectrophotometrical method was also used with the determining of the total concentration of albuterol including in the dispersed phase and the continuous phase of liposome. Absolute ethanol was added into the liposome suspension to disrupt the liposome completely and the encapsulated albuterol was completely dissolved in the solution. The absorbencies were measured by a UV-VIS Varian Cary 50 Tablet instrument equipped with a thermostated quartz cell at the wavelengths of 278 nm. The encapsulation efficiency (EE) was calculated with the formula:

EE(%)=(Cₜₒₜₐₗ−C_{free})/Cₜₒₜₐₗ×100

where C_{free} is the concentration of unencapsulated albuterol in the continuous phase of liposome dispersion/solution, and Cₜₒₜₐₗ is the total concentration of albuterol in the liposome dispersion/solution. The encapsulation efficiencies were 50-70%.

### Example 4

Preparation of Dehydrated Lipid Vesicles

This example illustrates the method for preparing liposomal compositions containing stabilizers and/or plasticizers for controlled release of active pharmaceutical ingredient.

A mixture of glycerin, DOPC or DOTAP, and active pharmaceutical ingredient (albuterol, 0.04 mmol) in the mole ratio of 1:10:20 was dissolved in 4 ml of ethanol. Liposomal albuterol dispersion was formed by injecting the lipid/active pharmaceutical ingredient/ethanol solution into 50 ml of the phosphate buffered saline pH 7.4. Liposomes thus formed were extruded through a 0.4 or a 0.2 or a 0.1 μm polycarbonate membrane to produce uniform size distribution of liposome vesicles. Lactose in the mol ratio of 200% to DOPC or DOTAP was added and dissolved into the liposome suspension, and the temperature immediately decreased by liquid nitrogen. The frozen ice of the liposome suspension was placed into the −50° C. lyophilizer chamber and vacuum applied to the chamber till the water was dried off, and the dehydrated lipid vesicles were formed with the lactose powder.

Transmission Electron Microscope (TEM) Observation of the Dehydrated Lipid Vesicles

The MLV and the SUV liposome solution were prepared using the same methods as mentioned above. The lipid concentration of all the liposomes was controlled at 10 mg/mL. The liposome vesicles were the empty and albuterol encapsulated liposomes. Before the TEM observation, 10% of glucose and 1% glycerin were added into the vesicle solutions, and then each of the vesicle suspensions were diluted to 100 fold with pure water. The diluted solutions were dropped on carbon coated 400 mesh copper electron microscope grids (SPI Supplies® Lot #1110207, Structure probe, Inc. West Chester Pa., USA) and the grids were frozen by liquid nitrogen. The refrigerated grid with liposome vesicle suspension was lypophilized at −40° C. in the chamber of the lyophilizer (FreeZon® 6 Liter Freeze Dry System, Labconco Corporation, USA) for 72 hours. Finally, the grids were sealed into a 10 mL glass injection vial, and the liposome vesicles on the grids remained to be observed by TEM (FEI, Tecnai G2 20 STEM, England).

Determination of Encapsulation Efficiency

The dehydrated lipid vesicle powder mixture was dissolved and suspended quantitatively in pure water. Then, 150 μL of freshly prepared liposomal sample was centrifuged with an Avanti J-E centrifuge (type JA-20, 17400×g, 6° C., and 20 min) through Microcon Y-10 Centrifugal Filter Devices (Millipore) with a cut-off value of 10,000 Dalton. The concentration of albuterol in the centrifuged solution was determined spectrophotometrically at 276 nm. This concentration represented the concentration of albuterol in the continuous phase of the liposome (non-encapsulated albuterol). The spectrophotometrical method was also used with the determining of the total concentration of albuterol including in the dispersed phase and the continuous phase of liposome. Absolute ethanol was added into the liposome suspension to disrupt the liposome completely and the encapsulated albuterol was completely dissolved in the solution. The absorbencies were measured by a UV-VIS Varian Cary 50 Tablet instrument equipped with a thermostated quartz cell at the wavelengths of 278 nm. The encapsulation efficiency (EE) was calculated with the formula:

EE(%)=(Cₜₒₜₐₗ−C_{free})/Cₜₒₜₐₗ×100

Where C_{free} is the concentration of unencapsulated albuterol in the continuous phase of liposome dispersion/solution, and Cₜₒₜₐₗ is the total concentration of albuterol in the liposome dispersion/solution.

### Example 5

The Retention and the Release of Albuterol in Liposome during Dialysis Equilibrium

The dehydrated lipids vesicle powder mixture was dissolved and suspended quantitatively in pure water. In order to evaluate the retention of albuterol in liposome in vitro, the equilibrium between the dialysis phase, the continuous phase (dispersion medium) and the dispersion phase (inner of liposomes) suspension were performed by membrane dialysis at 25° C. The release medium was Tris-buffered saline buffer solution (pH 7.4), which was the same buffer solution used to hydrate lipid-albuterol thin membrane during liposome preparation, so as to maintain the osmotic balance between the liposome in dialysis tube and the release medium. DOPC and DOTAP liposomes were chosen for the study, due to their relative high encapsulation efficiency.

6 mL of each liposome sample was transferred into dialysis membrane tube (molecular weight 12,000-14,000 Dalton cut-off; Spectrum Medical Industries, Los Angeles, Calif.) and placed in the temperature-controlled beaker containing 150 mL of TBS. The contents of the beaker were stirred at 50 rpm at the temperature of 37° C. through out the experiment. 5 mL of the dialysis medium was withdrawn from 150 mL of the medium in total at the time point of 15, 30, 45, 60, 90, 120, 150 and 180 min, every hour thereafter for 3 h and every 3 hour thereafter till 24 h. Each withdrawal was followed by replacement of fresh dialysis medium. The samples were assayed by a UV-VIS Varian Cary 50 Tablet instrument equipped with a thermostat quartz cell at the wavelength of 278 nm for the concentration of albuterol in the release medium (Cᵣₘ). At the same time point, 300 μL liposome in dialysis tubing was pipetted out. 150 μL was centrifuged with an Avanti J-E centrifuge (type JA-20, 17400×g, 6° C., 20 min) through Microcon Y-10 Centrifugal Filter Devices (Millipore) with a cut-off value of 10 kD. The concentration of albuterol in the centrifuged solutions was determined to be the concentration of albuterol in the continuous phase of liposomes at the instant time point (C_{free}). Another 150 μL liposome was determined to be the total concentration of albuterol in the liposome solutions (Cₜₒₜₐₗ) after appropriate dilution with ethanol. The difference between Cₜₒₜₐₗ and C_{free} was calculated as Cᵢₙ, which was regarded as the albuterol concentrations within the liposome at corresponding sampling time point.

The dialysis phase partition coefficient between continuous phase and dispersed phase of liposome (K_{free/in}=C_{free}/Cᵢₙ) and the dialysis phase partition coefficient between the continuous phase of the liposome and the release medium (K_{rm/free}=Cᵣₘ/C_{free}) were calculated.

After the sinking condition formed to the lipid vesicles, the K_{free/in}<10; stop the dialysis, and record the time, and test the Cₜₒₜₐₗ and C_{free} following the time, to calculate the release of the entrapped albuterol from the lipids vesicle in to the buffer. FIG. 2 shows the release of the entrapped albuterol from two kinds of the dehydrated lipids vesicles.

1. A pharmaceutical lipid composition for treatment of asthma by inhalation into a respiratory system, said composition comprising dehydrated lipid vesicles of a pharmaceutically acceptable vesicle preserver, a pharmaceutically acceptable lipid component and an active pharmaceutical ingredient wherein the pharmaceutically acceptable vesicle preserver includes plasticizers and/or stabilizers.

2. The composition of claim 1, wherein said vesicle preserver is chosen from pharmaceutical stabilizers and plasticizers selected from the group consisting of adipic acid and its derivatives or salts, ascorbic acid and its derivatives or salts, aspartic acid and its derivatives or salts, acetyltryptophan and its derivatives or salts, acetanilide and its derivatives or salts, aminoethy sulfonic acid and its derivatives or salts, alanine and its derivatives or salts, acacia, sodium bisulfite, sodium sulfite, arginine and its derivatives or salts, alginic acid and its derivatives or salts, benzoic acid and its derivatives or salts, isostearic acid and its derivatives or salts, inositol and its derivatives or salts, ethylenediamine and its derivatives or salts, erythorbic acid and its derivatives or salts, lysine and its derivatives or salts, cacao butter, castor wax, xathan gum, xylitol, citric acid and its derivatives or salts, glycine and its derivatives or salts, glycerin and its derivatives, gluconic acid and its derivatives or salts, glutamic acid and its derivatives or salts, creatinine, diisopropanolamine and its derivatives, diethanolamine and its derivatives, cyclodextrin, cystine, cysteine, dibutylhydroxytoluene, tartaric acid and its derivatives or salts, sucrose esters of fatty acids, stearic acid and its derivatives or salts, gelatin, lanolin, cetanol, gelatin, hydrolyzed gelatin, shellac, D-sorbitol, sorbitan esters of fatty acid, sorbica acid and its derivatives or salts, thioglycolic acid and its derivatives or salts, potassium thiocyanate, sodium thiomalate, thymol, medium chain fatty acid triglyceride, dextran, dextrin, vitamin E, calcium D-saccharate, tocopherol and its isomer, trometamol, nicotinamide, lactic acid and its derivatives or salts, lactose, carbamide, white soft sugar, histidine and its derivatives or salts, hydroxypropylcellulose, hydroquinone, phenylalanine, phenacetin, glucose, fumaric acid and its derivatives or salts, propylene glycol, heparin sodium, povidone, maleic acid and its derivatives or salts, malonic acid and its derivatives or salts, mannitol, methionine, sodium lauryl sulfate, malic acid and its derivatives or salts, hydrogenated oil, sesame oil, karion 83, diethylenetriaminepentaacetic acid and its derivatives or salts, dioctyl sodium sulfosuccinate, polydimethylsiloxane-silicone dioxide mixture, sorbitan esters of fatty acid, triacetin, castor oil, diethyl/dibutyl phthate, butylphtalylbutylglycolate, propylene glycol (1,2-propane diol), propylene glycol esters of fatty acids, polysorbate, polyoxyethylene polyoxypropylen glycol, macrogol, isopropyl myristate, cotton seed oil-soybean oil mixture, glyceryl monostearate, isopropyl linoleate, petrolatum, and mixtures thereof.

3. The composition of claim 2, wherein the composition has a ratio of said vesicle preserver to said lipid component from 0.1 to 40 mole % of the vesicle preserver and from 99.9 to 60 mole % of the lipid comnonent.

4. The composition of claim 1, wherein the active pharmaceutical ingredient is selected from the group consisting of ephedrine, ephedrine hydrochloride, albuterol, theophyline, salbutamol sulfate, salmefamol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rirniterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredient, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropiun bromide, cromolyn sodium, nedocromil and their respective pharmaceutically acceptable salts or esters, and mixtures thereof.

5. The composition of claim 4, wherein the composition has a mole ratio of active pharmaceutical ingredient to the lipid component from 0.1% to 200%.

6. The composition of claim 4, wherein albuterol is present in an amount between 0.1 to 300 mg/ml of the dehydrated lipid vesicles composition.

7. The composition of claim 1 which can be aerosolized into particles predominantly smaller than a mass median aerodynamic diameter of 10 μm.

8. A method of treating asthma by an inhalation route of administration to a person in need of such treatment a therapeutically effective amount of plasticized lipid composition consisting essentially of an active pharmaceutical ingredient, a vesicle preserver selected from a plasticizer, a stabilizer and mixtures thereof, and a lipid component aerosolized into aerosol particles having a mass median aerodynamic diameter smaller than 10 μm and providing a slow or controlled release of the active pharmaceutical ingredient into a respiratory system.

9. The method of claim 8 wherein the composition comprises 0.1 to 40 mole % of said stabilizers and/or plasticizer, 99.9 to 60 mole % of lipids, and the active pharmaceutical ingredient is from 0.01 to 200 mole % to the lipids.

10. The method of claim 8, wherein said active pharmaceutical ingredient is selected from the group consisting of ephedrine, ephedrine hydrochloride, albuterol, theophyline, salbutamol sulfate, salmefamol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rimiterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredientne, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropium bromide, cromolyn sodium, nedocromil and their respective pharmaceutically acceptable salts or esters, and mixtures thereof.

11. The method of claim 8 which employs albuterol in an amount from 0.1 to 300 mg/ml of the dehydrated lipid vesicle composition.

12. An inhalation method for treatment of respiratory system diseases by treating a person in need of such treatment with a therapeutically effective amount of an aerosolized dehydrated lipid vesicle composition consisting essentially of an active pharmaceutical ingredient and lipid components aerosolized into particles predominantly smaller than a mass median aerodynamic diameter of 10 μm by an inhalation route of administration.

13. The method of claim 12 wherein said active pharmaceutical ingredient is selected from the group consisting of ephedrine, ephedrine hydrochloride, albuterol, theophyline, salbutamol sulfate, salmefarnol, terbutaline, orciprenaline, fenoterol, clorprenaline hydrochloride, clorprenaline glycyrrhizinate, tulobuterol, 5-(4-amino-3,5-dichlorophenyl)-3-tert-butyloxazole, 5-(4-amino-3,5-dichlorophenyl)-3-tert-butyloxazole hydrochloride, clenbuterol hydrochloride, procaterol, salmeterol, hexoprenaline, mabuterol, formoterol, methoxyphenamine, tretoquinol, rimiterol, bitolterol, protokylol, reproterol, pirbuterol, fenspiride, ipratropine, isopropylscopolamine, aminophylline, diprophylline, choline theophyllinate, sodium cromoglicate, ketotifen, triprolidine, tranilast, ammonium chloride, potassium iodide, acetylcysteine, bromhexine hydrochloride, carbocisteine, ambroxol hydrochloride, guaifenesin, codeine, codeine phosphate, pholcodine, drotebanol, pentoxyverine citrate, chloperastine, benproperine phosphate, dextromethorphan hydrobromide, oxeladin, eprazinone, zipeprol, deoxopromethazine hydrochloride, fominoben, promolate, asverin, benzonatate, prenoxdiazine, noscactive pharmaceutical ingredientne, beclomethasone, betamethasone, budesonide, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, aldosterone, fluorometholone, flurandrenolone, halcinonide, hydrocortisone, meprednisone, methylprednisolone, paramethasone, prednisolone, prednisone, triamcinolone, metaproterenol sulfate, isoproterenol, adrenaline, norepinephrine, fluoromethasone, medrysone, fluticasone, atropine methyl nitrate, ipratropium bromide, cromolyn sodium, nedocromil and their respective pharmaceutically acceptable salts or esters, and mixtures thereof

14. The method of claim 12 which employs albuterol or other active pharmaceutical ingredients in an amount from 0.1 to 300 mg/ml of suspension of the dehydrated lipid vesicle composition.

15. A process of preparing a suspension of inhaleable or nebulizeable aerosol particles of sizes predominantly smaller than 10 μm, wherein the particles are dehydrated liposome vesicles, the process comprising providing dehydrated liposome vesicles having sizes less than 10 μm in an aqueous suspension; and inhaling or nebulizing the suspension under conditions which produce aerosol particles of a mass median aerodynamic diameter predominantly smaller than 10 μm.

16. The process of claim 15, wherein said particles comprise dehydrated lipid vesicles and/or micelles not larger than 5.0 μm, wherein said suspension is for treatment of asthma and consists essentially of lipid components and an active pharmaceutical ingredient or its salt or ester, suitable for delivery by inhalation into the respiratory system.
